(19) United States
(12) Patent Application Publication
(10) Pub. No.: US 2011/0238049 A1
(43) Pub. Date: Sep. 29, 2011

(54) Soft Tissue Laser

(75) Inventors: Reinhard Goerge, Heerbrugg (CH); Gottfried Rohner, Alstatten (CH)

(73) Assignee: IVOCLAR VIVADENT AG, Schaan (LI)

(21) Appl. No.: 13/048,073

(22) Filed: Mar. 15, 2011

(30) Foreign Application Priority Data
Mar. 23, 2010 (EP) 10157373.1

(51) Int. Cl. A61B 18/20

(52) U.S. Cl. 606/16

(57) ABSTRACT

A soft tissue laser with a display, a control device, a controllable laser light source, a first light-conducting element and a receptacle at the light-emitting side end of a first light-conducting element, at which different tips having a second light-conducting element each can be arranged in such a fashion that they can be removed again, which tips differ from one another in the diameters of the second light-conducting elements and/or in the amount by which the second light conductor axially exceeds the free end of the tip. The control device, in particular depending on the desired work process, determines the suitable tip and controls the laser light source in such a fashion that the power necessary for the work process selected is available at the free end of the second light-conducting element.

### CROSS-REFERENCE TO RELATED APPLICATIONS

This application claims the benefit of European Patent Application Serial No. 10157373.1, filed Mar. 23, 2010, which is hereby incorporated by reference in its entirety.

### TECHNICAL FIELD

The invention concerns a soft tissue laser, and more particularly relates to a soft tissue laser with changeable tips.

It has been known for some time to attach the tips of soft tissue lasers to the soft tissue laser in general in such a fashion that they can be removed, and as a result of the possibility of exchanging the tips, not only can problems concerning sterilization be prevented, but also the selection of the tip in the application to the micro-surgically desired case of application is possible. For example, the light-emission performance at the light-emitting end of the tip is altered by selecting a corresponding tip. Furthermore, it is also possible to reach areas to be treated which are located at a particularly deep position, if the front end is shaped to be offset or tapered.

Selecting tips for fibre-optic cables has been known for some time, for which reference is to be made, by way of example, to U.S. Pat. No. 3,831,017, which is hereby incorporated by reference. Even though the aspect of sterilization is not focused on in this solution, the possibility of exchanging the tip has become known, however in this solution different cable receptacles are used for different configurations.

In order to provide the desired effects, it has become known with soft tissue lasers to provide radiation with the help of a control device in a specific fashion, for instance via pulsed radiation of a previously defined power and a certain spectrum.

Furthermore, it has already been suggested to provide the exchangeable tips with a coding and/or to mark the tips in different colours in order to indicate their performance.

It is of disadvantage here that the colouring and the corresponding performance are not clearly obvious, which is of relevance in particular if the soft tissue laser is not applied on a daily basis, but rather only from time to time, such that the respective performances and their corresponding colours have to be looked up in each case.

It is a further problem that when changing the tips marked in different colours, the operator will typically rely on the laser performance which he assumes to be determined by the colour is really output, i.e. that the power is neither too high nor too low for the treatment of the soft tissue in a fashion corresponding to the wish of the operator. On the other hand, however, it is important to prevent accidental burns.

### SUMMARY OF THE INVENTION

Therefore, the invention provides a soft tissue laser which is distinctly safer in operation and can also be handled better from an ergonomic point of view. The soft tissue laser includes a display, a control device, a controllable laser light source, a first light-conducting element having a light-emitting side, and a receptacle at the light-emitting side end of the first light-conducting element, at which different tips having a second light-conducting element each can be arranged in such a fashion that they can be removed again, which tips differ from one another in the diameters of the second light-conducting elements and/or in the amount by which the second light conductor axially exceeds the free end of the receptacle tip, wherein the control device, depending on the desired work process, determines the suitable tip and controls the laser light source in such a fashion that the power necessary for the work process selected is available at the free end of the second light-conducting element.

In an embodiment of the present invention, it is intended to provide control by the operator, for instance via a monitor, in such a fashion that the desired power is preset. The operator will then receive the tip suitable for the performance desired—or a number of tips, according to his selection, which have different output ends but are to be grouped in the same level of performance—and will thus receive a secure-operation device which emits laser radiation for the treatment of soft tissue in the amount and in the fashion, i.e. concerning the pulse/pause ratio of the wave spectrum emitted etc., as he has set the device.

Now surprisingly secure-operation soft tissue lasers can be provided with the help of the measures in accordance with an embodiment of the present invention, for in an embodiment of the present invention the control is acting corresponding to the light power emitted adjacently to the working end, i.e. not, for instance, to the electrical power supplied.

The adaptation of power in accordance with an embodiment of the present invention can be put into practice in any suitable fashion. For example, the light power emitted can be measured after the corresponding tip has been mounted, and the electrical power of the laser can be adapted to this. It is also possible to provide each of the tips with a coding indicative of the reduction in power caused by the respective tip and thus giving a reliable indication of the cutting power emitted by the soft tissue laser.

It is particularly advantageous, in accordance with an embodiment of the present invention, if the stipulation by the operator concerning the light power is set as a priority by the control device and the currently used tip is also taken into consideration. For this purpose, a tip-recognition device can be used, for example with the help of a corresponding coding, which registers each tip used. This makes sure that the light power to be emitted will correspond to the operator's expectations.

If, for instance, the desired power set by the operator is 2 W and due to the tip selected half the light power is transmitted to the tip by means of the light-conducting element of the laser, for instance because the light-conducting surface of the tip is half the size of the light-conducing surface of the light-conducting element of the laser, the control device will automatically set an electrical power which corresponds to the light power of 2 W to be emitted at the light source. As a result, a light power of 1 W will be provided at the front end of the tip.

It is particularly favourable if not only the different diameters of the tip light-conducting device, but also the other parameters of the tip concerned, i.e. for instance the length of the tip, the offset portion of the front end, if necessary, or, for example, the length of the light-conducting element of the tip, are made clear with the help of a coding. The coding is preferably visually recognizable, for instance with the help of the colouring or shape of a retaining body of the tip element, as well as also preferably for this purpose with the help of a coding which is detectable via a sensor, such that pieces of information about the sort of tip can be supplied to the control device.

It is particularly favourable, in accordance with an embodiment of the present invention, that on the display at least two selectable work processes are depicted, in particular with regard to the tissue to be treated.

It is particularly favourable, in accordance with an embodiment of the present invention, that for at least one work process an in-contact mode or a non-contact mode can be selected.

It is particularly favourable, in accordance with an embodiment of the present invention, that on the display the shape of the tips—straight, inclined or curved—can be selected.

It is particularly favourable, in accordance with an embodiment of the present invention, that depending on the desired work process, the tip to be used is depicted on the display by the control device and the power of the laser light source is determined.

It is particularly favourable, in accordance with an embodiment of the present invention, that the power of the laser light source determined by the control device can be depicted on the display and/or can be altered manually.

It is particularly favourable, in accordance with an embodiment of the present invention, that the different tips have different codes of identification and that the code of identification determined can be depicted on the display.

It is particularly favourable, in accordance with an embodiment of the present invention, that the disposable tips have a code of identification each, and that the receptacle of the tips is provided with a detection device for the code of identification, which detection device supplies information about the sort of tip to the control device.

It is particularly favourable, in accordance with an embodiment of the present invention, that the code of identification is made up of a barcode and that the detection device is provided with a barcode reading device.

It is particularly favourable, in accordance with an embodiment of the present invention, that the detection device is provided with a sensor which recognises projections and/or recesses at the tip and that the code of identification is formed by at least one projection and/or one recess.

It is particularly favourable, in accordance with an embodiment of the present invention, that a diameter-detection device of the tip is provided, via which the diameter ratio between the diameter of the light-conducting element and that of the tip can be detected.

It is particularly favourable, in accordance with an embodiment of the present invention, that the control device is provided with a microphone and can be operated by means of voice input.

It is particularly favourable, in accordance with an embodiment of the present invention, that the control device is provided with a loudspeaker and indicates the settable combination of parameters by means of voice output.

It is particularly favourable, in accordance with an embodiment of the present invention, that the code of identification consists of colours and/or that the second light-conducting elements of the tips have a diameter of 200 μm, 300 μm, 400 μm or 600 μm.

It is particularly favourable, in accordance with an embodiment of the present invention, that the second light-conducting element exceeds the free end of the tip by 3 mm to 30 mm.

It is particularly favourable, in accordance with an embodiment of the present invention, that the laser light source has a power of 6 W to 12 W on average and/or that the laser light source creates light continually and/or in pulses.

It is particularly favourable, in accordance with an embodiment of the present invention, that in the transition area of the two light-conducting elements at least one optical transition element is positioned which bundles or extends the light beam of the first light-conducting element.

Further advantageous embodiments of the present invention are described in the appended claims and are taught in the following description.

### BRIEF DESCRIPTION OF THE DRAWINGS

Further advantages, details and features result from the following detailed description of embodiments of the invention with reference to the Figures, wherein:

FIG. 1 is a schematic view of a part of a soft tissue laser in accordance with the present invention in one embodiment of the invention, showing in particular the tip;

FIG. 2 is a perspective view of the soft tissue laser in accordance with FIG. 1, with a different tip mounted; and

FIG. 3 is a perspective view of the soft tissue laser in accordance with FIG. 1, with a third tip mounted.

### DETAILED DESCRIPTION

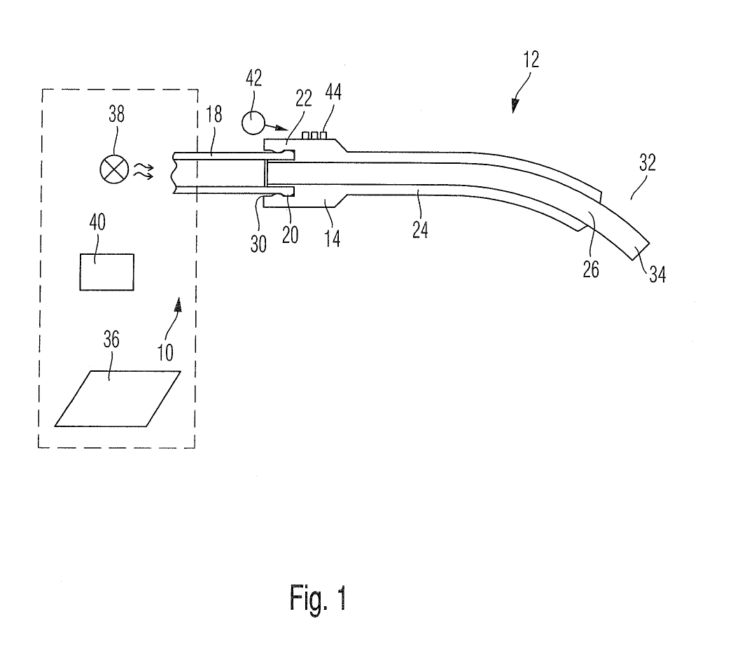

The soft tissue laser depicted in FIG. 1 has a tip 12 which is connected to the soft tissue laser 10 in general via a connection body 14. The soft tissue laser 10 has a light-conducting element 16 which is guided and protected inside a guide tube 18. The guide tube 18 has a boss 20 at its front end, which boss is provided for the positive coupling to the connection body 14. For this purpose, the connection body 14 has projections 22 pointing inwards at its end facing towards the guide tube 18, which projections 22 are schematically depicted in FIG. 1 and can flexibly grip over the boss 20 and can safely fix the tip 12 on the guide tube 18.

It is to be understood that any other way of coupling, for instance also via a bayonet joint, is possible without any problem and without exceeding the scope of the invention.

The tip 12 is provided with a shank 24 adjacent to the connection body 14, which shank 24 surrounds and protects a second light-conducting element 26. The light-conducting element 26 flush-contacts flatly the front end of the light-conducting element 16 at an interface 30 in such a fashion that a coupling efficiency of almost 100% is guaranteed. For this purpose, the plane surfaces of the light-conducting element 16 and the light-conducting element 26 facing each other are precisely positioned relative to one another, which is supported by the guidance of the light-conducting element 26 in the front end of the guide tube 18. The spring action of the projections 26 additionally presses the free end of the light-conducting element 26 symmetrically onto the free end of the light-conducting element 16.

The shank 24 of the tip 12 continues adjacently to the connection body 14 first coaxially with the light-conducting element 16. At the tip 12 depicted in the Figure, it is adjacently curved in the direction towards its front free end 32, with the curve radius corresponding to approximately five times the diameter of the light-conducting element 16.

As can be taken from FIG. 1, the second light-conducting element 26 points freely out of the shank 24, with the extent of the projection of the tip depicted corresponding to approximately three times the diameter of the light-conducting element 26. It is to be understood that the values indicated are to a great extent adaptable to the requirements.

Due to the projection, a particularly good exposure to laser light also of spaces accessible only with difficulty and narrow spaces is guaranteed. It is to be understood that the free end 34 of the second light-conducting element 26 can be protected in any suitable fashion, for instance with the help of a protective film which covers it.

In accordance with the present invention, the tip 12 can be exchanged against another tip voluntarily, with the diameter of the second light-conducting element 26 being different, with the extent of projection of the end 34 being different, or with the offset or curving or the straightness of the tip being of a different fashion. In accordance with the present invention, the tip 12 used is determined by a control device 36, which is depicted schematically in the fashion of a block diagram in FIG. 1, and which is positioned in the area of the housing of the soft tissue laser 10.

The control device 36 determines the suitable tip by itself. The light source 38 of the control device 36 is controlled in such a fashion, in accordance with the present invention, that the power necessary for the work process selected is available at the free end 34 of the second light-conducting element. For this purpose, the tip 12 is preferably depicted and indicated on a monitor 40 and is, if necessary, additionally recognised via a sensor 42. So the sensor 42 detects the amount of the diameter ratio of the diameter of the light-conducting element 26 and the diameter of the light-conducting element 16 and controls the light source 38 in the respectively suitable fashion.

It is to be understood that both the work process used and the tip to be used are depicted on the monitor 40 in any suitable fashion. If the sensor 42 is used, the tip used is preferably also visually depicted on the monitor at the same time, such that an operationally secure and ergonomic application of the soft tissue laser is possible.

It is to be understood that the tip 12, and of course any tip used, has an identification suitable for this purpose, for instance via a code of identification 44 which has been applied to the connection body 14 or to any other suitable section of the tip 12.

In a modified embodiment, not depicted here, the control device 36 has a microphone and is operable via voice input, with a loudspeaker preferably being provided for this purpose as well, which loudspeaker indicates the combination of parameters set by means of voice output, but which also offers, for instance, the combinations to be set by means of voice output.

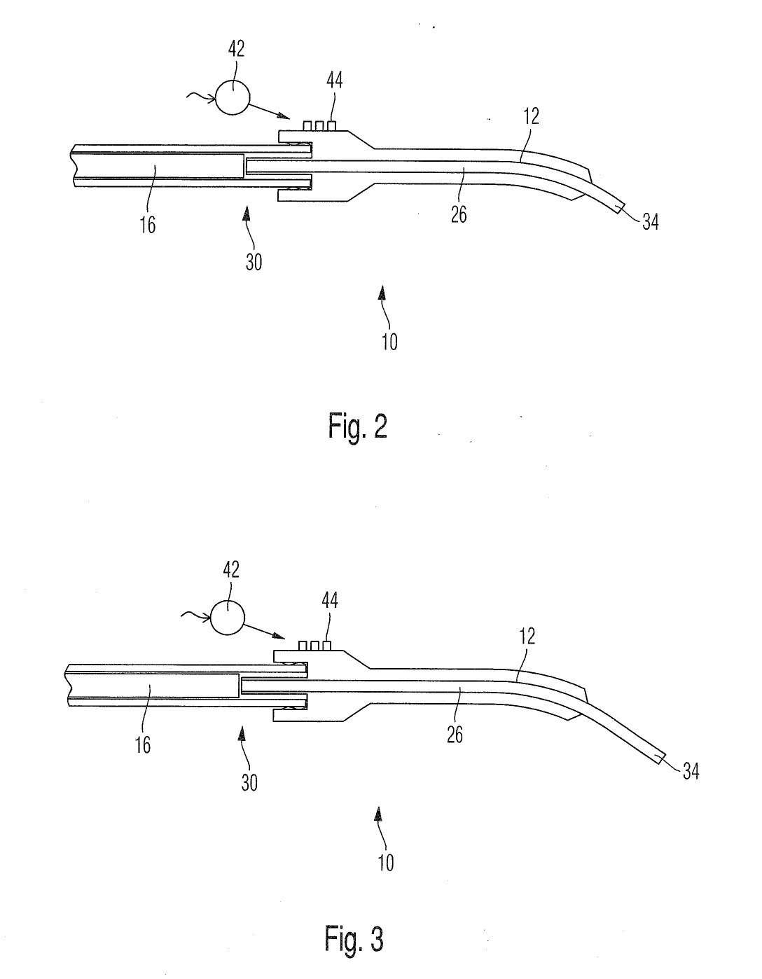

FIG. 2 shows a modified tip 12, with the soft tissue laser 10 otherwise corresponding to the soft tissue laser 10 in accordance with FIG. 1, such that it needs not be explained here in further detail. In this embodiment of the tip 12, the light-conducting element 26 is distinctly thinner. Its diameter is about half the diameter of the light-conducting element 16, such that in the area of the interface 30, a coupling of only 50% of the free surface of the light-conducting element 16 is possible. The light power emitted at the free end 34, therefore, corresponds to about 50% of the light power of the free end in accordance with FIG. 1.

This is made clear by means of a correspondingly modified code of identification 44, such that it is automatically recognized via the sensor 42 that a different tip 12 has been mounted.

Tip 12 in accordance with FIG. 3 differs from tip 12 in accordance with FIG. 2 by a longer projecting free end 34. This tip is therefore basically even better suited to provide the desired soft tissue laser light power in narrow spaces, with a careful handling being desirable due to the longer projecting free end 34.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. Soft tissue laser comprising:
a display,
a control device,
a controllable laser light source,
a first light-conducting element having a light-emitting side, and
a receptacle at the light-emitting side end of the first light-conducting element, at which different tips having a second light-conducting element each can be arranged in such a fashion that they can be removed again, which tips differ from one another in the diameters of the second light-conducting elements and/or in the amount by which the second light conductor axially exceeds the free end of the receptacle,
wherein the control device determines the suitable tip and controls the laser light source.

2. Soft tissue laser according to claim 1, characterized in that the control device determines the suitable tip and controls the laser light source based on a desired work process and wherein power necessary for the work process selected is available at the free end of the second light-conducting element.

3. Soft tissue laser according to claim 1, characterized in that on the display at least two selectable work processes are depicted, with regards to the tissue to be treated.

4. Soft tissue laser according to claim 1, characterized in that for at least one work process an in-contact mode or a non-contact mode can be selected.

5. Soft tissue laser according to claim 1, characterized in that on the display the shape of the tips comprising straight, inclined or curved can be selected.

6. Soft tissue laser according to claim 1, characterized in that depending on a desired work process, the tip to be used is depicted on the display by the control device and the power of the laser light source is determined.

7. Soft tissue laser according to claim 1, characterized in that the power of the laser light source determined by the control device can be depicted on the display and/or can be altered manually.

8. Soft tissue laser according to claim 1, characterized in that the different tips have different codes of identification and in that the code of identification determined can be depicted on the display.

9. Soft tissue laser according to claim 1, characterized in that the disposable tips have a code of identification each, and in that the receptacle of the tips is provided with a detection device for the code of identification, which detection device supplies information about the sort of tip to the control device.

10. Soft tissue laser according to claim 8, characterized in that the code of identification is made up of a barcode and in that the detection device is provided with a barcode reading device.

11. Soft tissue laser according to claim 8, characterized in that the detection device is provided with a sensor which recognises projections and/or recesses at the tip and in that the code of identification is formed by at least one projection and/or one recess.

12. Soft tissue laser according to claim 1, characterized in that a diameter-detection device of the tip is provided, via which the diameter ratio between the diameter of the light-conducting element and that of the tip can be detected.

13. Soft tissue laser according to claim 1, characterized in that the control device is provided with a microphone and can be operated by means of voice input.

14. Soft tissue laser according to claim 1, characterized in that the control device is provided with a loudspeaker and indicates the settable combination of parameters by means of voice output.

15. Soft tissue laser according to claim 8, characterized in that the code of identification consists of colours and/or in that the second light-conducting elements of the tips have a diameter of 200 μm, 300 μm, 400 μm or 600 μm.

16. Soft tissue laser according to claim 1, characterized in that the second light-conducting element exceeds the free end of the tip by 3 mm to 30 mm.

17. Soft tissue laser according to claim 1, characterized in that the laser light source has a power of 6 W to 12 W on average and/or in that the laser light source creates light continually and/or in pulses.

18. Soft tissue laser according to claim 1, characterized in that in the transition area of the two light-conducting elements at least one optical transition element is positioned which bundles or extends the light beam of the first light-conducting element.
